(19) United States
(12) Patent Application Publication
(10) Pub. No.: US 2013/0317342 A1
(43) Pub. Date: Nov. 28, 2013

(54) PATIENT SUPPORT APPARATUS, AND MEDICAL IMAGING APPARATUS WITH A PATIENT SUPPORT APPARATUS

(71) Applicant: Andrew Dewdney, Neunkirchen am Brand (DE)

(72) Inventor: Andrew Dewdney, Neunkirchen am Brand (DE)

(21) Appl. No.: 13/898,854

(22) Filed: May 21, 2013

(30) Foreign Application Priority Data
May 22, 2012 (DE) 102012208561.0

(51) Int. Cl. A61B 6/04

(52) U.S. Cl. CPC A61B 6/0407
USPC 600/410; 600/407; 5/601

(57) ABSTRACT

A patient support apparatus for supporting and/or transporting a patient is provided. The patient support apparatus may be used, for example in an imaging device. The patient support apparatus includes a patient support couch, on which the patient can be positioned for a medical imaging examination. A carrier unit is provided, on which the patient support couch is arranged so as to be moveable. The patient support apparatus also includes at least one drive unit for generating kinetic energy for a movement of the patient support couch in respect of the carrier unit. The at least one drive unit includes a pneumatic drive unit.

### CROSS REFERENCE TO RELATED APPLICATIONS

The present application claims priority to German Patent Office application No 102012208561.0 DE filed May 22, 2012, the entire content of which is hereby incorporated herein by reference.

### FIELD OF INVENTION

The present invention relates to a patient support apparatus, for in particular a medical imaging apparatus, for supporting and/or transporting a patient, including:
a patient support couch, on which the patient can be positioned for a medical imaging examination,
a carrier unit, on which the patient support couch is arranged so as to be moveable, and
at least one drive unit for generating kinetic energy for a movement of the patient support couch in respect of the carrier unit.

### BACKGROUND OF INVENTION

Patient support apparatuses of medical imaging apparatuses previously used electrical drive units in order to displace and/or move a patient support couch of the patient support apparatus. Particularly with magnetic resonance apparatuses, these electrical drive units can however only be operated in a magnetic field with a strength of less than 0.1 T.

With by contrast magnetic field strengths of magnetic resonance apparatuses of 1.5 T to 11.7 T, the electrical drive units must to this end be shielded and/or arranged outside of a region of the magnetic resonance apparatus which is penetrated by the magnetic field. To prevent further interferences and/or influences between the electrical drive units and the applied magnetic field, these electrical drive units were previously shielded and arranged fixedly within the patient support apparatus and in this way arranged with as maximal a distance as possible from a field of view (FOV) of the magnetic resonance apparatus.

### SUMMARY OF INVENTION

The object underlying the present invention is in particular to provide a patient support apparatus for a medical imaging apparatus, which allows for an arrangement of a drive unit within the patient support apparatus irrespective of a spatial expansion of a magnetic field. The object is achieved by the features of the independent claims. Advantageous embodiments are described in the subclaims

The invention is based on a patient support apparatus, for in particular a medical imaging apparatus, for supporting and/or transporting a patient, including:
a patient support couch, on which the patient can be positioned for a medical imaging examination,
a carrier unit, on which the patient support couch is arranged so as to be moveable, and
at least one drive unit for generating kinetic energy for a movement of the patient support couch in respect of the carrier unit.

It is proposed that the at least one drive unit includes a pneumatic drive unit, as a result of which it is advantageously possible to dispense with electrical and/or magnetic components, in particular moving electrical and/or magnetic components, of the drive unit, and in this way prevent an unwanted interference between the drive unit and an applied magnetic field of the medical imaging apparatus. In addition, a significant freedom of scope in respect of an arrangement of the drive unit within the patient support apparatus and/or a design of the patient apparatus irrespective of a spatial expansion of the applied magnetic field can also be achieved. In this context, a pneumatic drive unit should in particular be understood to mean a gas expansion motor unit, which is operated by means of a compressed gas, in particular compressed air. The medical imaging apparatus is preferably formed by a magnetic resonance apparatus and/or a combination apparatus with a magnetic resonance apparatus and a further medical imaging apparatus, such as for instance a positron emission tomography apparatus (PET).

It is furthermore proposed that the at least one drive unit is arranged directly between the carrier unit and the patient support couch. It may herewith feature the drive unit in a particularly space-saving manner in a region of the patient support apparatus with a short transmission path to a moving unit and/or a bearing unit/transmission unit of the patient support apparatus and in this way advantageously save on additional components and/or installation space. The at least one drive unit is herewith particularly advantageously arranged on the patient support couch and can be moved, during a movement of the patient support couch, together with the patient support couch in respect of the carrier unit.

In an advantageous development of the invention, it is proposed that the patient support apparatus comprises a subbase unit and at least one further drive unit for generating kinetic energy for a movement of the carrier unit in respect of the subbase unit. On account of the design of the patient support apparatus having at least two drive units, a movement of the patient and/or the patient support couch in respect of a change in movement and/or a change in speed can be optimized. For instance, one of the two drive units, together with a bearing unit/transmission unit, may be designed for a rapid positioning speed and a further of the two drive units, together with a further bearing unit/transmission unit, can be designed for a slow movement speed, in particular for a fine positioning, of the patient support apparatus.

If the at least one further drive unit includes a pneumatic drive unit, in particular a gas expansion unit, it is advantageously possible to dispense with electrical and/or magnetic components, in particular moving electrical and/or magnetic components, of the further drive unit and in this way prevent an unwanted interference between the further drive unit and an applied magnetic field of the medical imaging apparatus. Furthermore, a large freedom of scope in respect of an arrangement of the drive unit within the patient support apparatus and/or a design of the patient support apparatus can be achieved in this way irrespective of a spatial expansion of the applied magnetic field.

The at least one further drive unit is preferably herewith arranged directly on the carrier unit and/or the subbase unit, so that a particularly space-saving arrangement can be achieved within the patient support apparatus with a short transmission path to a moving unit and/or a bearing unit/transmission unit of the patient support apparatus and additional components and/or installation space can advantageously be saved in this way.

A particularly flexible positioning of the patient and/or of the patient support couch within a receiving area of a medical imaging apparatus, in particular of the magnetic resonance apparatus, can be achieved if the patient support apparatus comprises a control unit and the control unit controls the at least one drive unit and the at least one further drive unit independently of one another. In particular, the patient support couch can herewith be moved independently of a movement of the carrier unit.

It is furthermore proposed that the patient support apparatus comprises a gas line unit for a gas supply from a gas reservoir to at least one drive unit, as a result of which an advantageous gas supply can be achieved and the drive unit can be arranged irrespective of an arrangement and/or positioning of the gas reservoir. The gas reservoir is preferably arranged outside of the patient support apparatus, thereby additionally safeguarding the safety of the patient. The gas line unit preferably comprises at least one gas line, which includes a plastic line so that an unwanted impairment of a medical imaging examination, in particular a magnetic resonance examination, can be prevented.

If the patient support apparatus comprises a bearing unit/transmission unit, wherein the bearing and/or transmission unit and/or at least one drive unit is formed from a non-magnetizable material, an advantageous use of the patient support apparatus can be achieved within a magnetic resonance apparatus and an unwanted impairment of a magnetic field of the magnetic resonance apparatus can be prevented. The bearing unit/transmission unit and/or at least one drive unit is herewith particularly advantageously formed at least partially of stainless steel.

Furthermore, the invention is based on a medical imaging apparatus, in particular a magnetic resonance apparatus, with an inventive patient support apparatus.

It is proposed that the medical imaging apparatus includes a receiving region for receiving a patient positioned on the patient support apparatus, wherein the receiving region is penetrated by a magnetic field during operation of the medical imaging apparatus, and at least one drive unit, together with the patient support couch of the patient support apparatus, is arranged so as to be moveable within the receiving region. A particularly space-saving arrangement of the at least one drive unit can herewith be achieved and in this way a particularly short transmission path of kinetic energy to a bearing unit/joint unit of the patient support apparatus in order to move the patient support couch in respect of the carrier unit.

### BRIEF DESCRIPTION OF THE DRAWINGS

Further advantages, features and details of the invention result from the exemplary embodiment described below as well as with the aid of the drawings, in which:

FIG. 1 shows a medical imaging apparatus with an inventive patient support apparatus in a schematic representation.

### DETAILED DESCRIPTION OF INVENTION

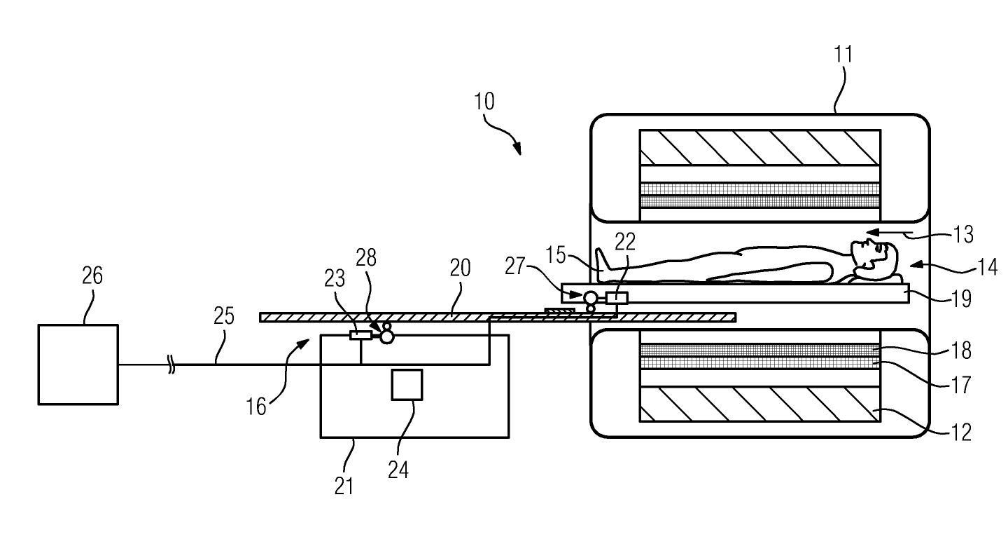

A medical imaging apparatus 10 formed by a magnetic resonance apparatus is shown in FIG. 1. Alternatively, the medical imaging apparatus 10 can to this end also include a combination apparatus comprising a magnetic resonance apparatus and a further medical imaging apparatus, such as for instance a PET apparatus.

The magnetic resonance apparatus includes a magnet unit 11 with a main magnet 12 for generating a strong and in particular constant main magnetic field 13. In addition, the magnetic resonance apparatus comprises a cylindrical receiving region 14 for receiving a patient 15, wherein the receiving region 14 is surrounded in a peripheral direction by the magnet unit 11. The patient 15 can be moved into the receiving region 14 by means of a patient support apparatus 16 of the magnetic resonance apparatus. The magnet unit 11 further comprises a gradient coil unit 17 for generating magnetic field gradients, which is used for local encoding during an imaging process. The magnet unit 11 furthermore comprises a cylindrical high-frequency coil unit 18 for exciting a polarization, which develops in the main magnetic field 13 generated by the main magnet 12. The magnetic resonance apparatus comprises a control unit (not shown in further detail) in order to control the main magnet 12, the gradient coil unit 17 and to control the high-frequency coil unit 18.

The patient support apparatus 16 is designed to support and/or transport the patient 15, in particular within the magnetic resonance apparatus. To this end, the patient support apparatus 16 comprises a patient support couch 19, on which the patient 15 is positioned for a medical imaging examination, in particular a magnetic resonance examination. Furthermore, the patient support apparatus 16 comprises a carrier unit 20 and a subbase unit 21, wherein the carrier unit 20 is arranged between the subbase unit 21 and the patient support couch 19.

The patient support couch 19 is arranged so as to be moveable in respect of the carrier unit 20. To this end, the patient support apparatus 16 comprises a first drive unit 22 for generating kinetic energy for moving the patient support couch 19 in respect of the carrier unit 20. The first drive unit 22 includes a pneumatic drive unit 22, which is formed by a gas expansion motor unit. The pneumatic drive unit 22 is arranged directly between the carrier unit 20 and the patient support couch 19, wherein the pneumatic drive unit 22 is herewith arranged on a side of the patient support couch 19 facing the carrier unit 20 and, during a movement of the patient support couch 19 can be moved together with the patient support couch 19 in respect of the carrier unit 20. As an alternative to the present exemplary embodiment, the patient support apparatus 16 can also comprise more than one first pneumatic drive unit 22. In addition, the first pneumatic drive unit 22 can also be arranged on the carrier unit 20, in particular on a side of the carrier unit 20 facing the patient support couch 19.

Thanks to the arrangement of the first pneumatic drive unit 22 on the patient support couch 19 the first pneumatic drive unit 22 is moved and/or displaced together with the patient support couch 19 during a movement and/or displacement of the patient support couch 19 into the receiving region 14 and/or within the receiving region 14 and thus within a region penetrated by the main magnetic field 13. In addition, the embodiment of the first pneumatic drive unit 22 as a gas expansion motor unit does not require any additional unit for shielding the first pneumatic drive unit 22, since the gas expansion motor unit has no electrical and/or magnetically embodied and moving motor components. An interference and/or unwanted influence between the first pneumatic drive unit 22 and the magnetic field 13 is reduced and/or prevented in this way.

The carrier unit 20 is also arranged to be moveable in respect of the subbase unit 21. To this end, the patient support apparatus 16 comprises a further drive unit 22 for generating kinetic energy so as to move the carrier unit 20 in respect of the subbase unit 21. The further drive unit 23 also includes a pneumatic drive unit 23, which is formed by a gas expansion motor unit. The further pneumatic drive unit 23 is preferably arranged between the subbase unit 21 and the carrier unit 20. In the present exemplary embodiment, the further pneumatic drive unit 23 is arranged on a side of the subbase unit 21 facing the carrier unit 20. As an alternative to this, the patient support apparatus 16 can also comprise more than one further pneumatic drive unit 23 embodied as a gas expansion motor unit and/or at least one further pneumatic drive unit 23 can also be arranged on the carrier unit 20, in particular on a side of the carrier unit 20 facing the subbase unit 21.

The embodiment of the further pneumatic drive unit 23 as a gas expansion motor unit no longer requires an additional unit for shielding the further pneumatic drive unit 23, since the gas expansion motor unit does not comprise any electrical and/or magnetically embodied and moving motor components. An interference and/or unwanted influence between the further pneumatic drive unit 23 and the magnetic field 13 is reduced and/or prevented in this way.

The patient support apparatus 16 further comprises a control unit 24 for controlling the first pneumatic drive unit 22, in particular the first gas expansion motor unit, and for controlling the further pneumatic drive unit 23, in particular the further gas expansion motor unit. The first pneumatic drive unit 22 and the further pneumatic drive unit 23 are controlled independently of one another by means of the control unit 24, so that a movement of the patient support couch 19 in respect of the carrier unit 20 is independent of a movement of the carrier unit 20 in respect of the subbase unit 21.

For a gas supply to the first pneumatic drive unit 22 and the further pneumatic drive unit 23, the patient support apparatus 16 comprises a gas line unit 25. Compressed air is in particular routed from a gas reservoir 26 to the pneumatic drive units by means of the gas line unit 25, wherein the gas supply to the pneumatic drive units 22, 23 is controlled by the control unit 24. The gas reservoir 26 is preferably arranged outside of the patient support apparatus 16 and also outside of a magnetic resonance room, within which the magnetic resonance apparatus is arranged. The gas line unit 25 in the present exemplary embodiment comprises gas lines, which are formed by plastic lines so that an unwanted interference with the magnetic field 13 can be prevented. The arrangement of the gas line unit 25 within the patient support apparatus 16 is only indicated by way of example in FIG. 1. The gas line unit 25 can essentially be arranged at any positions and/or regions of the patient support apparatus 16, provided a gas supply which is required for operation of the pneumatic drive units 22, 23 is ensured.

A bearing unit/transmission unit 27 of the patient support apparatus 16 is arranged between the carrier unit 20 and the patient support couch 19 so as to move the patient support couch 19 in respect of the carrier unit 20. Furthermore, a further bearing unit/transmission unit 28 of the patient support apparatus 16 is also arranged between the subbase unit 21 and the carrier unit 20 so as to move the carrier unit 20 in respect of the subbase unit 21. Kinetic energy and/or drive torque generated by the pneumatic drive units 22, 23 is transmitted in the process to the respective bearing unit/transmission unit 27, 28 and is converted there into a movement of the patient support couch 19 and/or a movement of the carrier unit 20. The further bearing unit/transmission unit 28 is designed such that a rapid and rough positioning of the patient support couch 19, which is moved together with the carrier unit 20 during operation of the further pneumatic drive unit 23, is possible within the receiving region 14. The first bearing unit/transmission unit 27 is by contrast embodied such that a fine adjustment of the position of the patient support couch 19 and/or of the patient 15 is possible. The first bearing unit/transmission unit 27 can to this end include a worm drive and/or further drive types which appear expedient to the person skilled in the art.

The individual components of the first pneumatic drive unit 22 and the further pneumatic drive unit 23 as well as the individual components of the first bearing unit/transmission unit 27 and also the individual components of the further bearing unit/transmission unit 28 are formed from a non-magnetizable material. In addition, these components can also be formed from stainless steel. An unwanted interference is herewith prevented during operation of the magnetic resonance device.

1. A patient support apparatus for supporting and/or transporting a patient, the patient support apparatus comprising:
a patient support couch, on which the patient can be positioned for a medical imaging examination,
a carrier unit, on which the patient support couch is arranged so as to be moveable, and
at least one drive unit for generating kinetic energy for a movement of the patient support couch in respect of the carrier unit,
wherein the at least one drive unit includes a pneumatic drive unit.

2. The patient support apparatus as claimed in claim 1, wherein the at least one drive unit is arranged directly between the carrier unit and the patient support couch.

3. The patient support apparatus as claimed in claim 2, wherein the at least one drive unit is arranged on the patient support couch and can, during a movement of the patient support couch, be moved together with the patient support couch in respect of the carrier unit.

4. The patient support apparatus as claimed in claim 1, further comprising a subbase unit and at least one further drive unit for generating kinetic energy for a movement of the carrier unit in respect of the subbase unit.

5. The patient support apparatus as claimed in claim 4, wherein the at least one further drive unit includes a pneumatic drive unit.

6. The patient support apparatus as claimed in claim 4, further comprising a control unit which controls the at least one drive unit and the at least one further drive unit independently of one another.

7. The patient support apparatus as claimed in claim 1, further comprising a gas line unit for supplying gas from a gas reservoir to at least one drive unit.

8. The patient support apparatus as claimed in claim 1, further comprising at least one bearing unit/transmission unit, wherein the bearing unit/transmission unit and/or at least one drive unit is formed from a non-magnetizable material.

9. The patient support apparatus as claimed in claim 1, further comprising at least one bearing unit/transmission unit, wherein the bearing unit/transmission unit and/or at least one drive unit is formed at least partially from stainless steel.

10. A medical imaging apparatus, comprising:
a patient support apparatus as claimed in claim 1.

11. The medical imaging apparatus as claimed in claim 10, wherein the medical imaging apparatus is a magnetic resonance apparatus.

12. The medical imaging apparatus as claimed in claim 10, comprising a receiving region for receiving a patient positioned on the patient support apparatus, wherein during operation of the medical imaging apparatus the receiving region is penetrated by a magnetic field,
wherein at least one drive unit is arranged so as to be moveable together with the patient support couch within the receiving region.
